(19) United States
(12) Patent Application Publication
(10) Pub. No.: US 2007/0237834 A1
(43) Pub. Date: Oct. 11, 2007

(54) Zinc Zeolite for the Treatment for Diaper Rash (Diaper Dermatitis)

(75) Inventor: SHYAM K. GUPTA, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, SCOTTSDALE, AZ (US)

(21) Appl. No.: 11/760,466

(22) Filed: Jun. 8, 2007

Related U.S. Application Data
(63) Continuation-in-part of application No. 10/605,191, filed on Sep. 14, 2003.
(63) Continuation-in-part of application No. 10/418,495, filed on Apr. 18, 2003, now abandoned.

(51) Int. Cl. A61K 33/06; A61K 33/24; A61K 33/30; A61K 33/32; A61K 33/34; A61P 17/00

(52) U.S. Cl. 424/630; 424/639; 424/642; 424/646

(57) ABSTRACT

The present invention provides treatment of infants and incontinent adults related to diaper rash, also known as diaper dermatitis, by the application of divalent metal complexes of zeolites, such as zinc zeolite, and compositions thereof.

This is a continuation-in-part of U.S. patent application Ser. No. 10/605,191; filed on Sep. 14, 2003. Additional disclosures in related fields by the present inventor include; Gupta, U.S. Patent Application pre-Grant publications 20050276761 and 20060045860; and U.S. patent application Ser. No. 11/684,702 (filed Mar. 13, 2007).

The present invention discloses certain divalent metal complexes of zeolite, and cosmetic or pharmaceutical compositions that contain said zeolites, for the treatment of diaper dermatitis (diaper rash).

Baby skin is especially sensitive to environmental and dietary conditions. “Baby bottom” is unusually moist from frequent urination, which can promote bacterial growth that is known to cause skin irritation and infection. Also, excessive amounts of lipase and protease enzymes, and their metabolic products such as ammonia and fatty acids, are also released during frequent defecation by baby, which are further known to cause skin irritation and allergic skin rashes.

One of the most common skin problems with infants relates to diaper rash, also known as diaper dermatitis. One study conducted with infants less than two years of age concluded that almost two-thirds of all infants suffer from diaper rash of some degree. Approximately 10 percent of all infants can have their diaper rash classified as being moderate, with another 5 percent of the infants having diaper rash, which could be classified as severe. The primary contributors to the development of diaper rash have long been thought to be infant urine and feces. For example, infants under two months of age can urinate up to 20 times per day. Thereafter, infants can urinate up to 8 times a day. In addition, infant defecation typically occurs several times a day. It had been theorized that the breakdown of the urine to yield ammonia primarily contributed to the formation of diaper rash by increasing the alkalinity of the skin. However, more recent studies have concluded that the primary contributor to the development of diaper rash is a feces. As opposed to the alkaline pH associated with urine, feces typically exhibit an acidic pH due to bile. In fact, studies have shown that diaper rash is more prominent in the presence of feces than in the presence of urine, thereby providing a plausible explanation for the problems with diaper rash associated with infants who have diarrhea or frequent stools.

Diaper rash may predispose an infant to irritation and infection. The two most common types of infection are those associated with yeast, and bacteria. The most common yeast infection is caused by Candida albicans. Meanwhile, the most common bacterial infection is caused by Staphylococcus aureus.

However, merely keeping the area clean and dry does not protect the irritated skin from the chemical irritation associated with the by-products of infant urine and feces. U.S. Pat. No. 5,436,007 (Hartung et al.) discloses a skin lotion composition containing a linear polydimethylsiloxane polymer, a non-ionic emulsifier, consisting of polyoxyethylene sorbitan fatty acid esters, sorbitan fatty acid esters, polyoxyethylene alcohols, or polyoxyethylene fatty ethers aloe vera, an alkoxylated ether/ester, sodium citrate, citric acid, a blend of propylene glycol, diazolidinyl urea, methyl paraben and propyl paraben, and water. Most preferably the buffering system results in the lotion having a pH of about 5.2, which neutralizes acidic and basic by-products of urine and fecal matter. The lotion is claimed to be useful in the protection and treatment of diaper rash. However, as can be noted by anyone skilled in the art that Hartung disclosure provides only partial solution, i.e. the pH control of skin, of diaper rash problem.

An example of a diaper rash product is British Patent No. 1,357,731. That patent discloses a unique powder composition, which can be incorporated into a hydrophobic ointment. A buffer system is provided to buffer the composition at a pH of from 5.5 to 7.5, and preferably from 6 to 7. That patent discloses that a citric acid/sodium citrate buffering system does not have superior buffering capacity when compared with other buffering systems. This patent further discloses that “succinic acid/sodium succinate has 30 percent more buffering capacity than a citric acid/sodium citrate” buffer system. Thus this patent differs from U.S. Pat. No. 5,436,007 in that it claims use of a powder formulation having buffering capacity in the alkaline range of pH 5.5 to 7.5, and teaches away from the use of citric acid buffering system in view of the preferred use of a succinic acid buffer.

Another example of the prior art is U.S. Pat. No. 4,556,560 (Buckingham). This patent discloses and claims use of lipase inhibiting agents, such as the water soluble metallic salts including zinc chloride, in the treatment of diaper rash. This patent purports to treat diaper rash by inhibiting the deleterious effects of the enzyme lipase action on the skin, said inhibition being achieved by incorporating a inhibitory agent of said lipase action into a barrier like carrier, said carrier having the characteristics of being relatively hydrophobic in nature thereby forming an effective barrier to the skin against urine and feces.

U.S. Pat. No. 4,996,238 (Matravers) discloses a skin protective composition exhibiting enhanced water repellency and skin conditioning effects and contains aliphatic waxes and hydrophobic silicones. Matravers' specifically discloses and claims the use of an admixture consisting of a fatty acid admixed with one or more hydrophobic silicones. As can be noted by anyone skilled in the art that Matravers teaches only one aspect, i.e. the reduction of contact of skin with moisture, for diaper rash control.

It has recently come to be understood that the initial stages of some types of diaper rash are the result of skin irritation caused by contact with digestive enzymes present in infant feces, particularly trypsin, chymotrypsin and elastase. These enzymes are proteolytic enzymes produced in the gastrointestinal tract to digest food. In infants, the feces tend to be watery and they contain, among other materials such as bacteria, some amounts of undegraded digestive enzymes. These enzymes, if they remain in contact with the skin for any appreciable period of time have been found to cause an irritation that is uncomfortable and can predispose the skin to infection by microorganisms. U.S. Pat. Nos. 6,331,295 and 5,869,033 (Schulz) disclose compositions comprising an amount of organophilic clay effective to inactivate irritating fecal proteolytic enzymes dispersed in a pharmaceutically acceptable non-toxic dermatological vehicle. A fabric incorporating organophilic clay, preferably dispersed in a matrix of a super absorbent polymer is useful for preparing diapers for infants that can help to prevent skin irritation by fecal enzymes. As can be noted by anyone skilled in the art that Schulz teaches only one aspect, i.e. the adsorption of inflammatory enzymes, for diaper rash control. U.S. Pat. Nos. 3,935,862 and 4,273,786 refer to compositions containing amino acid compounds to inhibit the formation of ammonia and therefore treat diaper rash.

Skin rash caused by dermatitis, often referred to as diaper rash, has always been a problem encountered by the users of disposable absorbent articles, such as diapers, incontinence articles, sanitary towels, training pants etc. Therefore, one of the biggest needs for these users is a solution to this type of skin rash problem. The main factor which influences the development of skin rash is the contact of the skin with the wet body exudates, directly or for example contained in the absorbent article. Especially when the water content is high, skin rash can occur easily. Manufacturers of diapers and skin care products have developed various products over the past decades that help reduce the occurrence of diaper rash (or skin rash). The main focus thereby has been to reduce the exposure of the skin to the body exudates. This is for example done by introduction to the diaper of absorbing or better absorbing materials. The amount of water, which is in contact with the skin, is thus reduced. Other products, which are developed to address the skin-rash problem, reduce the exposure of the skin to certain ingredients of the body exudates. An example of such ingredients of the exudates is bacteria, which can infect the skin and thus start off or aggravate the skin rash. For example, lotions have been developed which can form a barrier between the skin and the body exudates. Also, anti-inflammatory compositions can be applied to the skin or absorbent article. However, still one of the most heard complaints amongst users of absorbent articles such as diapers is the persistence of skin or diaper rash, despite the numerous products on the market which can be applied to prevent diaper or skin rash. It has been discovered that yet another factor can set off or aggravate skin rash, namely the presence in the body exudates of various enzymes, especially lipase enzymes. When the skin is exposed to lipase enzymes, these enzymes can affect the lipids of the skin. U.S. Pat. No. 3,961,486 teaches the use of adipic acid to reduce the lipase enzyme activity and to reduce the skin rash. It is also known that bile salts are present in the body exudates. These bile salts are known to emulsify the lipase enzymes in the body, which ensures that the lipase enzymes are capable of performing on the lipid-water interface. It has been found that these bile salts still have an emulsifying function once outside the body, in the body exudates. They aid the lipase enzyme, which is present in the body exudates by attacking the lipids in the outer layer skin, exposed to the body exudates. U.S. Patent Application 20030035785 (Palumbo et al.) discloses that triester compounds similar to lipids or the lipids of the skin in particular can function as enzyme substrates, which, when acted upon by a hydrolyzing esterase enzyme, such as lipase enzymes, will be hydrolyzed resulting in the release of free acids. Firstly, the presence of these acids will lower the pH of the area where the esters where topical applied to. This will amount to inactivation of all or most enzymes present in this area, in the body exudates, such as the lipase enzymes, protease enzymes. Secondly, the esterase or lipase enzymes are ‘de-activated’, because rather than hydrolyzing the esters, such as lipids, of the skin, they hydrolyze the alternative substrate, the triester compounds of the invention. Palumbo et al. also disclose that the bile salts mentioned above can be inactivated (and thereby the lipase can be deactivated) when the bile salts are reacted with specific cationic compounds. Thus, the use of a combination of the cationic compounds and the triester compounds has an enhanced and elongated effect on the skin-rash or lipolytic dermatitis, according to Palumbo et al.

The skin of infants is known to be highly sensitive, particularly to chemical substances. One common skin problem of infants is diaper dermatitis; more commonly called “diaper rash.” FDA has defined “Diaper rash” as an inflammatory skin condition in the diaper area (perineum, buttocks, lower abdomen, and inner thighs) caused by one or more of the following factors: moisture, occlusion, chaffing, continued contact with urine or feces or both, or mechanical or chemical irritation [21 CFR Section 347.3 (1990)], and that definition will be used herein. The FDA has also indicated that mild diaper rash appears as simple erythema and that more severe conditions may be accompanied by papules, vesicles, oozing, and ulceration. Adults (e.g., incontinent adults) may also suffer from diaper rash. The FDA allows claims to be made that the following substances are useful as skin protectants provided, among other things, that those substances are used at FDA-specified concentration levels: mineral oil, dimethicone, zinc oxide, allantoin, calamine, kaolin, petrolatum, white petrolatum, cod liver oil, lanolin, talc, topical starch, aluminum hydroxide gel, cocoa butter, glycerin, shark liver oil, zinc acetate, and zinc carbonate, all of which will be referred to herein as “active ingredients for protecting skin” [21 CFR Section 347.10 (1983 and 1990)]. As used herein, the terms “protecting skin,” “protecting the skin,” and “protecting human skin” are synonymous and each include protecting and/or treating skin in connection with various indications involving the skin, including diaper rash; minor burns; cuts; scrapes; sunburn; chaffed, chapped, cracked, or wind-burned skin or lips; skin irritation; and oozing and/or weeping of skin caused by poison ivy, poison oak, and/or poison sumac. For example, assuming all the other requirements are met, the FDA will allow a claim to be made that a composition containing dimethicone is useful for treating diaper rash if the dimethicone concentration is from 1 percent wt (percent by weight) to 30 percent wt. A similar claim can be made for a composition containing zinc oxide if the zinc oxide concentration is from 1 percent wt to 40 percent wt. A similar claim can be made for a composition containing mineral oil if the mineral oil concentration is from 50 percent wt to 100 percent wt. As used herein, the term “treating diaper rash” includes treating an existing diaper rash condition or preventing a diaper rash condition or both. Compositions that may contact the skin and may contain zinc oxide, and/or mineral oil, and/or silicon dioxide (silica), and/or dimethicone or other silicone compounds, some of which compositions may be in the form of aerosols or sprays and some of which compositions may be used for treating diaper rash, include those compositions referred to in U.S. Pat. Nos. 2,843,522; 3,770,648; 3,935,862; 4,043,077; 4,196,218; 4,273,786; 4,278,658; 4,329,366; 4,389,418; 4,514,383; 4,556,560; 4,569,839; 4,574,082; 4,672,074; 4,725,438; 4,800,076; 4,816,254; 4,842,593; 4,847,071; 4,911,932; 4,933,330; 4,938,960; 4,996,238; 4,996,239; 5,043,359; 5,085,856; 5,137,714; 5,208,031; 5,210,102; 5,232,691; 5,234,689; 5,266,318; 5,362,488; 5,389,204; 5,436,007; 5,527,519; 5,543,135; 5,545,673; 5,558,872; 5,573,753; 5,576,006; 5,603,863; 5,616,331; 5,635,191; 5,643,588; 5,652,274; 5,662,937; 5,665,426; 5,730,993; 5,733,895; 5,744,146; 5,756,082; 5,756,110; 5,762,945; 5,776,440; 5,834,290; 5,861,143; 5,861,144; 5,861,146; 5,863,522; 5,869,061; 5,869,062; 5,869,071; 5,874,094; 5,885,599; 5,914,101; 5,939,053; 5,945,211; 5,958,397; 5,961,961; 5,962,441; 5,965,137; 5,965,610; 5,968,531; and 5,972,359.

As can be noted by anyone skilled in the art that the above disclosures teach only one aspect, i.e. the use of skin protectants, for diaper rash control. U.S. Pat. No. 3,770,648 refers to substantially non-aqueous quick-breaking aerosol foaming compositions containing silicone compounds (e.g., dimethyl silicone fluids), foamable organic liquids (e.g., mineral oil), and a high vapor pressure propellant system (e.g., fluorocarbons). Example 2 of the patent refers to what is called “DESITIN.RTM. Baby foam,” containing zinc oxide (about 4.2 percent wt), mineral oil (about 71.7 percent wt), a silicone, and FREON 12 fluorocarbon propellant. Desitin is a very popularly marketed diaper rash composition. However, it is based on only one aspect, i.e. use of skin protectant ingredients, for diaper rash control. Even the most recently filed disclosures, such as U.S. Patent Application 20010006666 (Harbeck), 20030082223 (Healy et al.), U.S. Pat. No. 5,762,945 (Ashley), and U.S. Pat. No. 6,419,963 (Niazi) utilize fatty compositions to merely provide a moisture barrier for diaper rash prone skin.

DE102005014370 discloses a topical product for the prevention and treatment of diaper dermatitis that contains betaine.

The present invention provides, surprisingly and unexpectedly, a comprehensive treatment for diaper rash by certain divalent metal complexes of zeolites, such as zinc zeolite, that encompasses the following aspects: (1) deactivation of lipase and protease enzymes on skin surface to remove the source of irritation, (2) trapping of acidic and alkaline chemicals deposited on skin from body exudates and enzyme activity to reduce the immediate skin reaction, (3) neutralization of bacteria, yeast, and fungus for preventing secondary infection and other such complications, and (4) absorption of moisture for reducing excess skin surface moisture. It is not known at this time exactly how a single ingredient, such as the divalent metal complex of zeolite of the present invention, can provide all of the above benefits, or provides a comprehensive treatment of diaper dermatitis. The utility of the present invention, irrespective of the above lack of knowledge, is both unprecedented and unexpected. It is, however, known at this time that it is not the potential antibacterial action of said zeolites that provides the above combination benefits for the treatment of diaper dermatitis.

Zeolites are a group of crystalline aluminosilicates that have a porous structure with a cavity. The preparation and properties of these zeolites are described in detail in U.S. Pat. No. 2,882,243, for example. Generally, the preparation involves combining aqueous solutions that are sources of silica, alumina and sodium to produce a gel that crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300 C provides anhydrous zeolite.

Zeolites have the following properties that can be highly useful for topical delivery of cosmetic and pharmaceutical compositions: (1) Zeolites have high adsorptive capacity for water and many organic compounds including toxic metals and enzymes (which makes them useful for many other applications such as water purification, waster water treatment, and chemicals refining/purification), (2) Zeolites are available in certain pore sizes that can be used for self-warming or non-warming cosmetic and pharmaceutical compositions, (3) Zeolites can be made anionic or cationic, which can be used for controlled-release of certain cosmetic and pharmaceutical ingredients via ion-pair mechanisms, (4) Zeolites have a very large surface area that can nearly achieve a nano-particle distribution of organic molecules attached to its vast surface area, (5) Zeolites can also be made in cations other than sodium or potassium, and (6) Zeolites do not absorb into the skin, which is useful for topical delivery of cosmetic and pharmaceutical compositions that are electronically attached to such zeolite surfaces for their controlled or slow delivery over a period of time.

However, many of the prior art applications of zeolites have centered upon their chemical catalysis, heat releasing, or trapping of small molecular weight ingredients. Zeolites also have outer surface area, in addition to such inner pore surface areas.

Zeolites can be made with both specific pore structures and bound cations that have found applications in various self-warming cosmetic compositions. U.S. Pat. No. 3,250,680 (Menkart et al.) discloses applications of Zeolites for the preparation of self-heating toothpaste and other such compositions. This utilizes only the heat releasing property of zeolites.

U.S. Pat. No. 4,626,550 (Hertzenberg) discloses certain personal care products such as lotions and creams that are prepared using Zeolite A that contains sodium and potassium.

EP1749514 discloses a dental composition comprising a silver zinc zeolite, at least one monomer having at least one ethylenically unsaturated group, and a polymerization initiator system. Silver appears to be the active metal agent in these compositions.

CN1857308 discloses a zinc replenisher with control released zinc and its preparation process and use. The zinc replenisher is zeolite containing active zinc in 1.5-10 wt percent. The preparation process includes the adsorption and ion exchange reaction between zinc salt and zeolite, filtering, drying and crushing to obtain zeolite containing active zinc. Supporting zinc onto zeolite, which possesses excellent biocompatibility, excellent gastrointestinal mucous membrane affinity, regular pore canal structures and cage spaces, high surface activity and great specific surface area, can obtain controlled releasing of zinc and raise utilization of zinc. The control released zinc preparation may be used as zinc replenisher for human body and animal.

KR960003801B discloses a nitrate ion containing antibacterial zeolite and antibacterial resin composition. This antibacterial zeolite contains nitrate ion of 0.1-15, silver of 0.1-20, copper or zinc of 0.5-20, and calcium or magnesium of 0.5-15. This is prepared from Na substituted zeolite and Ag, Zn, Cu, Ca, Mg salts of nitric acid. And antibacterial resin contains 0.05-60 of antibacterial zeolite.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various musculoskeletal problems.

U.S. Pat. No. 6,274,128 (Bergman et al.) discloses an essentially anhydrous hair conditioning composition that comprises of zeolites of specific pore size larger than the critical diameter of a water molecule and both the carrier molecules and the hair conditioner molecules that have molecular diameters larger than the largest average pore size of the micro porous materials. As is clearly evident, such constraints are not convenient or commercially achievable at a reasonable cost.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect.

U.S. Application 20010016201 (Janchitraponvej) discloses a yet another self-heating application of an anhydrous rinse-out hair care composition utilizing zeolites.

In self-warming formulations based on Zeolites, the pore size specification is typically very small, from 3 to 10 angstroms in diameter, as is the ratio between sodium and potassium cations bound to silicate anions of such zeolites. These formulations release heat upon contact with water. Water penetrates the pores of such Zeolites and hydrates the interior silicate atoms of Zeolite agglomerates. Such interaction of zeolite with water releases the heat of hydration. However, for moisture absorptive benefits, such as for a “baby bottom” cosmetic skin protectant product, the pore structure of zeolites is not important, as both the inner and outer surface of zeolites possesses water absorptive property. The divalent metal derivatives of zeolites, such as zinc and magnesium zeolite, are most useful for such dual-purpose (i.e. both water absorptive and skin protectant) benefits, as further disclosed in the present invention.

The monovalent metal complexes of zeolites, such as those disclosed by Raaf (U.S. Pat. No. 4,525,343), do not cause the treatment of diaper dermatitis.

Similarly, Gioffre et al. (U.S. Pat. No. 4,826,676) disclose certain anticariogenic and anticalculus compositions containing zeolitic zinc cations. However, Gioffre et al. do not disclose any diaper dermatitis treatment benefits of the same.

Dobrodzsi (U.S. Pat. No. 6,638,521) disclose oral liquid mucoadhesive compositions containing divalent metal zeolite, such as magnesium zeolite. However, Dobrodzsi claimed no diaper rash preventive or curative benefits.

Zeolites can also be made in cations other than sodium or potassium. U.S. Pat. No. 6,357,678 (Hu et al.) discloses preparation of zinc zeolites by a very difficult multi-step process. U.S. Pat. No. 6,605,267 (Lee et al.) disclose process for making metal zeolites with quaternary ammonium compounds, useful as chemical catalysts. U.S. Pat. No. 6,084,142 (Yao et al.) discloses the preparation of a zinc zeolite, and its application in petroleum cracking process. U.S. Pat. No. 6,177,374 (Pradhan et al.) discloses the preparation of silicon, zinc and aluminum zeolites, and their application in petroleum cracking process. Yao and Pradhan do not disclose any cosmetic or diaper rash applications of such zeolite derivatives. U.S. Pat. No. 6,479,427 (Anthony at al.) and U.S. Pat. No. 5,502,240 (Pugach) disclose titanium zeolites and their application in petroleum cracking process. U.S. Pat. No. 5,772,917 (Kynast et al.) discloses a cesium zeolite that is luminescent. U.S. Pat. No. 6,106,797 (Muller et al.) discloses titanium or vanadium zeolites useful for accelerating oxidation reactions. U.S. Pat. No. 6,008,389 (Grosch et al.) discloses titanium and vanadium zeolites useful as catalysts for the preparation of epoxides, in particular propylene oxide, from olefins, hydrogen and oxygen. U.S. Patent Application 20030035763 (Vergani et al.) discloses the use of iron and manganese zeolites in the purification of organometallic compounds utilizing such zeolite's adsorptive properties. U.S. Patent Application 20030024856 (Surana et al.) discloses a yet another application of zeolite's adsorptive properties in removing odors.

U.S. Patent Application 20020127402 (Green et al.) discloses the antimicrobial applications of silver ions attached to zeolites by ion-exchange methods. Green et al have not disclosed the attachment of any organic molecules to zeolites by ion-exchange method.

It is worthy of note that although zeolites with many different cations, such as titanium, zinc, manganese, iron, quaternary ammonium, and copper have been disclosed, any applications of such metal zeolites in cosmetic or pharmaceutical applications have not been disclosed. It is further worthy of note that both titanium and zinc are well known in their oxide state as sun block agents that have been used in sunscreen compositions now for several years. It is further worthy of note that zinc salts are known for their antimicrobial, skin protectant, and anti-irritant properties. Zinc oxide, for example, is a FDA-approved drug ingredient for skin protectant compositions. Zinc acetate, zinc chloride, zinc carbonate, zinc ricinoleate, and zinc sulfate have all been used for antiseptic, astringent, and skin protective compositions, as mentioned in The Merck Index, 12th Edition (1996). The divalent salts of zinc, in general, have antibacterial and skin protectant properties. The skin protectant or any other skin beneficial benefits of divalent zinc zeolites have not been reported in the prior art.

It would thus be of much potential commercial and consumer interest to develop applications of divalent metal zeolites, such as zinc and titanium zeolites, in cosmetic or pharmaceutical compositions. The present inventor has disclosed some of these novel applications (Gupta, U.S. Patent Application pre-Grant publications 20050276761; 20060045860; and U.S. patent application Ser. No. 11/684,702).

This lack of prior art knowledge is of special note, since zeolites with enhanced ion-exchange capacity are well known (U.S. Patent Application 20010053741, Mikko et al.; U.S. Pat. No. 5,935,891; Prior). U.S. Pat. No. 6,503,740 (Alther et al.) discloses zeolites treated with an organic modification compound such as quaternary amines, pyridinium compounds, and phosphonium amines that are useful for water treatment applications. U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman). Additionally, U.S. Pat. No. 4,620,929 (Hofmann) and U.S. Pat. No. 3,935,067 (Thayer) teach the use of expanded clay or plastic materials in combination with bentonite, which advantageously exhibits moisture retention characteristics. Topical skin beneficial benefits of such zeolites have not been reported in the prior art.

The present invention discloses a cosmetic or pharmaceutical composition comprising; (i) a divalent metal complex of zeolite, and, wherein (ii) said composition is for the treatment of diaper dermatitis. Said compositions can include a cosmetically or pharmaceutically acceptable carrier or base. The divalent metal complex of zeolite is selected from a group of divalent metal complexes of aluminosilicates that are either in hydrated or in an anhydrous form. The divalent metal complex of zeolite is further selected from zinc zeolite, or molybdenum zeolite, or calcium zeolite, or manganese zeolite, or copper zeolite, or magnesium zeolite, or a combination thereof. The divalent metal complex of zeolite of choice is zinc zeolite. The said composition can include an amino acid, which is selected from glycine, or alanine, or valine, or leucine, or isoleucine, or serine, or threonine, or tyrosine, or cysteine, or methionine, or aspartic acid, or asparagine, or glutamic acid, or glutamine, or arginine, or lysine, or histidine, or phenylalanine, or tryptophane, or proline, or hydroxyproline, or beta-alanine, or beta-aminoisobutanoic acid, or homocysteine, or homoserine, or ornithine, or citrulline, or 5-amino levulinic acid, or picolinic acid, or a combination thereof.

The said composition can further include skin cleansers, surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

The cosmetically acceptable carrier is selected in the form of a lotion, cream, gel, spray, thin liquid, body splash, mask, serum, solid cosmetic stick, lip balm, shampoo, liquid soap, bar soap, bath oil, paste, salve, collodion, impregnated patch, impregnated strip, skin surface implant, diaper, and other such cosmetically or pharmaceutically acceptable topical forms.

The cosmetically or pharmaceutically acceptable base is selected from traditional water and oil emulsions, suspensions, colloids, micro-emulsions, clear solutions, suspensions of nanoparticles, emulsions of nanoparticles, or anhydrous compositions. Said cosmetically or pharmaceutically acceptable preferred carrier is a diaper. Said cosmetically or pharmaceutically acceptable preferred base is a cream or a powder.

The present invention also discloses a method of treating diaper dermatitis skin condition comprising topically applying to the skin, for a period of time and in an amount sufficient to effect changes in the dermis, of a composition comprising a divalent metal complex of zeolite. Said divalent metal complex of zeolite of choice is zinc zeolite. Said divalent metal complex of zeolite can be further selected from zinc zeolite, or molybdenum zeolite, or manganese zeolite, or copper zeolite, or a combination thereof. The said composition can include an amino acid, which is selected from glycine, or alanine, or valine, or leucine, or isoleucine, or serine, or threonine, or tyrosine, or cysteine, or methionine, or aspartic acid, or asparagine, or glutamic acid, or glutamine, or arginine, or lysine, or histidine, or phenylalanine, or tryptophane, or proline, or hydroxyproline, or beta-alanine, or beta-aminoisobutanoic acid, or homocysteine, or homoserine, or ornithine, or citrulline, or 5-amino levulinic acid, or picolinic acid, or a combination thereof. Said composition can further include a cosmetically or pharmaceutically acceptable carrier or base. Said base is selected from traditional water and oil emulsions, suspensions, colloids, micro-emulsions, clear solutions, suspensions of nanoparticles, emulsions of nanoparticles, or anhydrous compositions. Said carrier is selected from a lotion, cream, gel, spray, thin liquid, body splash, mask, serum, solid cosmetic stick, lip balm, shampoo, liquid soap, bar soap, bath oil, paste, salve, collodion, impregnated patch, impregnated strip, skin surface implant, diaper, and other such cosmetically or pharmaceutically acceptable topical forms.

The present invention also discloses simple in-situ preparation of divalent metal complexes of zeolites. A very simple process can prepare the divalent metal zeolites by the ion-pair exchange of a zinc salt (such as zinc chloride, zinc sulfate, zinc nitrate, zinc acetate, zinc gluconate, zinc EDTA, etc.) with a zeolite, as illustrated in Equation 1, 2, and 3 for the preparation of zinc zeolite.
Zinc Chloride+Zeolite=Zinc Zeolite+Sodium (potassium) Chloride (Equation 1).
Zinc Acetate+Zeolite=Zinc Zeolite+Sodium (potassium) Acetate (Equation 2).
Zinc Gluconate+Zeolite=Zinc Zeolite+Sodium (potassium) Gluconate (Equation 3).

It should be noted that zeolites contain sodium and potassium cations that can be exchanged with other cations. It is commonly known that the exchange efficiency is in the following order for some metals: Ba>Pb>Cd>Zn>Cu>K>Na>Li. The exchange amount is determined by the exchange capacity of such zeolites, which is usually expressed as milli-equivalents (meq) of a cationic composition to per gram weight of zeolite. A zeolite with 1.0 meq per gram exchange capacity, for example, can exchange 0.068 grams of zinc chloride per gram of such zeolite. This is calculated as follows. The molecular weight of zinc chloride is 136.3. Thus, 136.3 grams of zinc chloride equals 1000 milli-equivalents (or, 1 mole equivalent), or 0.136 grams of zinc chloride equals one milli-equivalent. Since each zinc chloride molecule has two chlorine atoms that can undergo exchange, only half the equivalent amount of zinc chloride will thus be needed to exchange monovalent cations (such as sodium or potassium) in that zeolite. Thus, only 0.06815 grams of zinc chloride will be needed to exchange with one gram of zeolite for a complete exchange (i.e. 136.3/1000/2=0.06815). In practice, total exchange is not required. Typically, only 10 to 50 percent of all available monovalent cations need to be exchanged. In another example, 0.0917 grams of zinc acetate (molecular weight 183.4) will be needed to completely exchange one gram of zeolite that has one meq per gram of exchange capacity with two acetate anions to be exchanged (i.e. 183.4/1000/2=0.092).

Moreover, the exchange reactions of the present invention can be carried out in anhydrous systems. This offers a great advantage for the preparation of anhydrous zeolites containing divalent cations. The preparation of divalent metal zeolites by ion exchange is usually carried out in an aqueous medium followed by their dehydration at elevated temperatures, during which many divalent metal zeolite cage structures collapse. The methodology of the present invention circumvents this problem and permits the preparation of anhydrous zeolites with divalent cations without requiring a high temperature dehydration step, since anhydrous forms of zeolites can now be exchanged with divalent cations in an anhydrous medium according to the teachings of the present invention. Such anhydrous zeolites are useful for their high water absorption property.

In actual preparative process, a solution of zinc derivative in water or another solvent or solvent mixture is stirred with zeolite. The in-situ process, as shown in Equation 1 and 2, thus forms zinc zeolite. Other divalent derivatives of zinc can also be used, such as zinc acetate, zinc carbonate, etc. in Equation 1 or 2. In addition to zinc, virtually any other monovalent, divalent, or polyvalent metal can be complexed with zeolite surface by such ion-pair bonds to prepare metal-zeolite ion-pairs. Examples include, but not limited to, copper zeolite, manganese zeolite, magnesium zeolite, calcium zeolite, iron zeolite, and such.

The compositions of the present invention can include additional agents to either adsorb or neutralize acidic or alkaline chemicals on skin surface. The examples include, but not limited to clays, silica gels, water absorbent organic polymers, water retentive cellulose, starch, and inulin derivatives, carbomers, dehydro xanthan, cotton, paper fibers, ion exchange resins, chitosan, psyllium husk, algin, agar, carrageenan, gelatin, pectin, locust bean gum, gum arabica, xanthan gum, gellan gum, purified seaweeds (granulated Spirulina), alginate salts, rice bran husk, oat flour, oat protein, colloidal oat protein, soya flour, soya protein, wheat flour, wheat protein, milk powder, milk protein, egg powder, egg protein, casein, rice flour, corn starch, modified starches, rice starch, tapioca starch, inulin, hydrolyzed inulin, soya fibers, cotton fibers, cellulose, modified celluloses, sugars, modified carbohydrates, fenugreek fibers, silk fibers, various clays, zeolites, anhydrous zeolites, fumed silica, porous silica, alumina, various plant gums, and combinations thereof.

The compositions of the present invention can include anti-inflammatory agents. It is to be noted that a mixture of two or more anti-inflammatory compositions, especially those that belong to different biochemical mechanism classes, is more beneficial than corresponding equal weight amounts of a single ingredient. This is due to various different biochemical mechanisms by which such anti-inflammatory compositions provide their beneficial effect. A number of both synthetic and natural compositions have thus become available; some of such examples follow (the biochemical mechanism of their action is indicated in the parentheses). Ginger Root, or Zingiber Officinale Root Extract (COX-2 inhibitor), Galanga, or Alpinia Officinarum Extract (LOX-5 inhibitor), Turmeric, or Curcuma Longa Root Extract (Superoxide inhibitor), Mango Ginger, or Curcuma amada (Unknown mechanism), Capsicum, or Capsicum Annuum Extract (Substance P inhibitor, Vasodilation, Superoxide inhibitor), Clove Family, or Syzygium Aromaticum Extract (COX-1, COX-2 inhibitor), Evodia, or Evodia Rutaecarpa Fruit Extract, (COX-2 inhibitor), Boswellia, or Boswellia Serrata Extract (LOX-5 inhibitor), SAMe, or S-Adenosylmethionine (Catecholamine metabolism), Eucomis, or Eucomis L″Herit (COX-1 inhibitor), Celastrus, or Celastrus orbiculatus (COX-1 inhibitor), Tithonia, or Tithonia diversifolia (Cytokine inhibitor), Kochia, or Kochia Scoparia Extract (COX-2 inhibitor), Scoparia, or Scoparia dulcis Extract (Analgesic), Qiang Huo, or Notopterygium incisum (COX-1, LOX-5 inhibitor), Cinnamon, or Cinnamonum cassia (Nitric oxide scavenger), Mexican Bamboo, or Polygonum cuspidatum (Nitric Oxide scavenger), Ogon , Baikal Scullcap, or Scutellaria baicalensis (COX-2 inhibitor), Coptis, Xianglian, or Coptis chinensis (Nitric oxide inhibitor), Psoralea, Rumex, Baccharis, Feverfew, Vitis, Stephania (unknown mechanisms), and Corydalis, or Corydalis Turtschaninovii Root Extract (Analgesic).

The compositions of the present invention can include antioxidant ingredients. Most of such antioxidants additionally possess anti-inflammatory and antimicrobial properties. A combination of antioxidants, or antioxidant and anti-inflammatory mixture, is more effective than a single antioxidant or anti-inflammatory composition on an equal weight basis due to antioxidant and anti-inflammatory cascade mechanisms. It is well known that antioxidants belong to various chemical classes, such as polyphenols, carotenoids, flavonoids, and such. Some examples follow. (Chemical class is indicated in parentheses.) Rutin (flavone), Quercetin (flavone), Hesperidin (flavone), Diosmin (flavone), Mangiferin (xanthone), Mangostin (xanthone), Cyanidin (carotenoid), Astaxanthin (carotenoid), Xanthophyll (carotenoid), Lycopene (carotenoid), carotene (carotenoid), resveratrol, (polyphenol), tetrahydrocurcumin (polyphenol), rosmarinic acid (polyphenol), ellagic acid (polyphenol), hypericin (polyphenol), chlorogenic acid (polyphenol), oleuropein (polyphenol), lipoic acid (disulfide), glutathione-oxidized (disulfide), cystine (disulfide), N-acetyl-cystine (disulfide), glutathione- reduced (sulfhydryl), cystein (sulfhydryl), and N-acetyl-cysteine (sulfhydryl).

The compositions of the present invention can include additional cosmetically or pharmaceutically beneficial ingredients can also be included in the compositions of the present invention, which can be selected from, but not limited to skin cleansers, surfactants (cationic, anionic, non-ionic, amphoteric, and zwitterionic), skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

The compositions of the present invention can include skin protectant drug agents, which can be selected from, but not limited to Allantoin, petrolatum, glycerin, dimethicone, urea, calamine, cocoa butter, kaolin, zinc oxide, zinc acetate, zinc carbonate, and combinations thereof.

The compositions of the present invention can include UVA/UVB sunscreen composition, which can be selected from Titanium dioxide, Zinc oxide, Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, and PABA, and combinations thereof.

The compositions of the present invention can include collagen and elastin synthesis boosters, which can be selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia Mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica Granatum), Chlorogenic acid (Vaccinium vulgaris), Oleuropein (Olea europaea), a-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof.

### EXAMPLES

The following examples are presented to illustrate presently preferred practice thereof. As illustrations they are not intended to limit the scope of the invention. All quantities are in weight percent.

### Example 1

### Preparation of Zinc Zeolite from Zinc Chloride

(1) Zeolite, Type 4A 20.0 (2) Zinc chloride 1.36 (3) Glycerin 78.64. Procedure. Mix (2) and (3) to a clear solution. Add (1) and mix. The mixture contains Zinc zeolite (100 percent zeolite exchanged), made by the in-situ ion-pair exchange.

### Example 2

### Preparation of Zinc Zeolite from Zinc Acetate

(1) Zeolite, Type 4A 40.0 (2) Zinc Acetate 0.18 (3) Glycerin 59.82. Procedure. Mix (2) and (3) to a clear solution. Add (1) and mix. The mixture contains Zinc zeolite (5 percent zeolite exchanged), made by the in-situ ion-pair exchange.

### Example 3

### Preparation of Benzalkonium Zeolite

(1) Zeolite, Type 4A 10.0 (2) Benzalkonium chloride (50 percent solution) 2.0 (3) Propylene Glycol 88.0. Procedure. Mix all ingredients together. Benzalkonium zeolite is formed in-situ.

### Example 4

### “Baby Bottom” Cream Composition

(1) Zinc Zeolite 3.0 (2) Glycerin 49.0 (3) Sodium Potassium Aluminosilicate (Zeolite A3) 20.0 (4) Anti-irritant Composition 1.0 (The antiaging composition is an equal weight mixture of Tetrahydrocurcumin, Niacinamide salicylate, Horse Chestnut extract, Glutathione, and Carnosine) (5) Silicone Wax 27.0. Procedure: Mix (1), (2), and (3) to a thin paste. Add all other ingredients and mix. A white paste is obtained.

### Example 5

### “Baby Wash” Composition

(1) PEG-633.5 (2) Vitamin A Palmitate 0.1 (3) Vitamin E Acetate 0.1 (4) Actiplex Botanicals 0.1 (5) Phenoxyethanol 0.5 (6) Liquapar 0.2 (7) Zinc Zeolite 10.5 (8) Zeolite (as moisture absorbent) 28.0 (9) Sodium Lauryl Sulfoacetate 8.5 (10) Sodium Cocoyl Isethionate 14.0 (11) Benzalkonium zeolite 4.0 (12) Fragrance 0.5. Procedure: Mix all ingredients in a homogenizer mill. A paste is obtained.

### Example 6

### Self-Warming and Water Absorbent Baby Body Butter Composition

(1) Castor 0il 20.8 (2) Mango Butter 2.0 (3) Cocoa Butter 4.0 (4) Beeswax 3.5 (5) Stimu-Tex 0.2 (6) Avocado Butter 1.0 (7) Shea Butter 4.0 (8) Sweet Almond Oil 2.0 (9) Grapeseed Oil 2.0 (10) Dimethicone 5.0 (11) Hydrogenated Soybean Oil 6.0 (12) Sesame Oil 0.9 (13) Tinoguard TT 0.2 (14) Phenoxyethanol 0.5 (15) Propyl Paraben 0.2 (16) Aloe Vera (In Oil) 4.0 (17) Vitamin E Acetate 0.1 (18) Vitamin A Palmitate 0.1 (19) Zeolite (Atofina Nk30np) 35.0 (20) Zinc Zeolite 8.5. Procedure: Mix all ingredients and heat at 60 to 70 C. Cool to room temperature. A butter-like material is obtained.

### Example 7

### “Baby Bottom” Mild Foaming Cleanser Composition

(1) PEG-6 32.9 (2) Vitamin A Palmitate 0.1 (3) Vitamin E Acetate 0.1 (4) Phenoxyethanol 0.5 (5) Propyl Paraben 0.3 (6) Shea butter 1.0 (7) Apricot Kernel Oil 0.5 (8) Grapeseed Oil 0.5 (9) Kiwi Fruit Seed Oil 0.5 (10) Mango butter 0.5 (11) Zeolite (4A) 30.0 (12) Disodium Lauryl Sulfosuccinate 14.0 (13) Sodium Cocoyl Isethionate 7.0 (14) Polyquaternium-59 Zeolite 2.0 (15) Zinc Zeolite 10.0 (16) Esculoside 0.5 (17) Darutoside 0.5 (18) Vitamin K 0.1. Procedure. Mix all ingredients to a thick paste.

### Example 8

### Water Absorbent “Baby Bottom” Butter Composition

(1) Grapeseed Oil 15.8 (2) Mango Butter 0.5 (3) Cocoa Butter 0.5 (4) Beeswax 1.0 (5) Aloe butter 0.2 (6) Avocado Butter 0.5 (7) Shea Butter 0.5 (8) Vitamin E 0.1 (9) Grapeseed Oil 2.0 (10) Dimethicone 1.0 (11) Hydrogenated Soybean Oil 35.0 (12) Sesame Oil 0.9 (13) Tinoguard TT 0.2 (14) Phenoxyethanol 0.5 (15) Propyl Paraben 0.2 (16) Zeolite (Atofina Nk30np) 28.0 (17) Zinc Zeolite 5.0 (18) Behentrimonium Zeolite 2.0 (19) Esculoside 0.5 (20) Darutoside 0.5 (21) Vitamin K 0.1 (22) Corn starch 5.0. Procedure: Mix all ingredients and heat at 60 to 70 C. Cool to room temperature. A butter-like material is obtained.

### Example 9

### “Baby Bottom” Emollient Paste

(1) Paraffin Wax 25.0 (2) Propyl Paraben 0.1 (3) Cetyl Alcohol 1.0 (4) GMS-SE 4.0 (5) Stearic Acid 3.0 (6) Polawax 5.0 (7) Deionized Water 47.8 (8) Methyl Paraben 0.2 (9) Aloe vera 0.2 (10) Triethanolamine 0.5 (11) Dimethicone/Dimethiconol 2.0 (12) Zinc Zeolite 10.0 (13) Tetrahydrocurcumin 0.2 (14) Esculin 0.5 (15) Boswellia serrata 0.5. Procedure. Mix ingredients (1) to (11) and heat at 80 to 90 C to a uniform mixture. Cool to 40 to 50 C. Add all other ingredients and mix. Cool to room temperature. An off-white paste is obtained.

### Example 10

### Diaper Rash Balm

(1) Castor Oil 50.9 (2) Mango Butter 8.0 (3) Cocoa Butter 6.0 (4) Beeswax 2.0 (5) Zinc Zeolite 12.0 (6) Copper Zeolite 0.5 (7) Titanium Dioxide 1.0 (8) Shea Butter 4.0 (9) Sweet Almond Oil 2.0 (10) Grape Seed Oil 2.0 (11) Hydrogenated Soybean oil 8.0 (12) Sesame Oil 0.9 (13) BHT 0.2 (14) Phenoxyethanol 0.5 (15) Propyl Paraben 0.2 (16) Aloe vera 0.5 (17) Vitamin E Acetate 0.1 (18) Vitamin A Palmitate 0.1 (19) Vitamin K 0.1 (20) Darutoside 0.5 (21) Oleuropein 0.5. Procedure. Mix (1) to 15) and heat at 60 to 70 C to a slurry. Cool to 40 to 50 C and all other ingredients. Cool to room temperature.

### Example 11

### Diaper Rash Powder

(1) Corn Starch 75.0 (2) Zinc Zeolitel 4.0 (3) PEG-65.0 (4) Tetrahydrocurcumin 0.5 (5) Vitamin K-10.5 (6) Dimethicone 5.0. Procedure. Mix (1) and (2). Premix (3) to (6) and add to main batch and mix. A powder composition is obtained.

1. A cosmetic or pharmaceutical composition comprising; (i) a divalent metal complex of zeolite and, (ii) wherein said composition is for the treatment of diaper dermatitis.

2. A composition according to claim 1, wherein said composition includes a cosmetically or pharmaceutically acceptable carrier or base.

3. A composition according to claim 1, wherein divalent metal complex of zeolite is selected from a group of divalent metal complexes of aluminosilicates that are either in hydrated or in an anhydrous form.

4. A composition according to claim 1, wherein divalent metal complex of zeolite is selected from zinc zeolite, or manganese zeolite, or copper zeolite, or molybdenum zeolite, or a combination thereof.

5. A composition according to claim 1, wherein divalent metal complex of zeolite is zinc zeolite.

6. A composition according to claim 1, wherein divalent metal complex of zeolite is made in-situ from a monovalent metal salt of a zeolite and a divalent metal donor.

7. A composition according to claim 1, wherein said diaper dermatitis is diaper rash.

8. A composition according to claim 1, wherein said composition includes an amino acid, which is selected from glycine, or alanine, or valine, or leucine, or isoleucine, or serine, or threonine, or tyrosine, or cysteine, or methionine, or aspartic acid, or asparagine, or glutamic acid, or glutamine, or arginine, or lysine, or histidine, or phenylalanine, or tryptophane, or proline, or hydroxyproline, or beta-alanine, or beta-aminoisobutanoic acid, or homocysteine, or homoserine, or ornithine, or citrulline, or 5-amino levulinic acid, or picolinic acid, or a combination thereof.

9. A composition according to claim 2, wherein a cosmetically acceptable carrier is selected in the form of a lotion, cream, gel, spray, thin liquid, body splash, mask, serum, solid cosmetic stick, lip balm, shampoo, liquid soap, bar soap, bath oil, paste, salve, collodion, impregnated patch, impregnated strip, skin surface implant, diaper, and other such cosmetically or pharmaceutically acceptable topical forms.

10. A composition according to claim 2, wherein the cosmetically or pharmaceutically acceptable base is selected from traditional water and oil emulsions, suspensions, colloids, micro-emulsions, clear solutions, suspensions of nanoparticles, emulsions of nanoparticles, or anhydrous compositions.

11. A composition according to claim 2, wherein said cosmetically or pharmaceutically acceptable carrier is a diaper.

12. A composition according to claim 2, wherein said cosmetically or pharmaceutically acceptable base is a cream.

13. A composition according to claim 2, wherein said cosmetically or pharmaceutically acceptable base is a powder.

14. A method of treating diaper dermatitis skin condition comprising topically applying to the skin, for a period of time and in an amount sufficient to effect changes in said skin condition, of a composition comprising a divalent metal complex of zeolite.

15. A method according to claim 14, wherein said divalent metal complex of zeolite is zinc zeolite.

16. A method according to claim 14, wherein said divalent metal complex of zeolite is further selected from zinc zeolite, or manganese zeolite, or molybdenum zeolite, or copper zeolite, or a combination thereof.

17. A method according to claim 14, wherein an amino acid, selected from glycine, or alanine, or valine, or leucine, or isoleucine, or serine, or threonine, or tyrosine, or cysteine, or methionine, or aspartic acid, or asparagine, or glutamic acid, or glutamine, or arginine, or lysine, or histidine, or phenylalanine, or tryptophane, or proline, or hydroxyproline, or beta-alanine, or beta-aminoisobutanoic acid, or homocysteine, or homoserine, or ornithine, or citrulline, or 5-amino levulinic acid, or picolinic acid, or a combination thereof, is included.

18. A method according to claim 14, wherein said composition includes a cosmetically or pharmaceutically acceptable carrier or base.

19. A method according to claim 18, wherein said base is a cream.

20. A method according to claim 18, wherein said carrier is a diaper.
